(19) United States
(12) Patent Application Publication
(10) Pub. No.: US 2015/0065902 A1
(43) Pub. Date: Mar. 5, 2015

(54) COLUMNAR FLOW GAS SAMPLING AND MEASUREMENT SYSTEM

(71) Applicant: Capnia, Inc., Redwood City, CA (US)

(72) Inventors: Pedro E. DE LA SERNA, San Jose, CA (US); Anthony D. WONDKA, San Ramon, CA (US); Robert BLANTE, Hayward, CA (US)

(21) Appl. No.: 14/474,019

(22) Filed: Aug. 29, 2014

Related U.S. Application Data
(60) Provisional application No. 61/872,270, filed on Aug. 30, 2013.

(51) Int. Cl. A61B 5/08; A61B 5/00

(52) U.S. Cl. CPC A61B 5/082; A61B 5/6819
USPC 600/532

(57) ABSTRACT

A breath analysis device is described which minimizes mixing of gases between one section of a breath and another section of breath. In particular for example, when sampling and analyzing the end-tidal section of exhaled gas, the system may avoid mixing that can occur inside the device, between the end-tidal sample and the gases before and after the end-tidal sample. The system accomplishes this with an ultra-low uniform cross section fluid pathway, which includes componentry with ultra-low dead space.

### CROSS-REFERENCE TO RELATED APPLICATION

This application claims the benefit of U.S. Provisional Application No. 61/872,270, filed Aug. 30, 2013, the content of which is incorporated herein in its entirety.

### FIELD OF THE INVENTION

The disclosure relates to the field of diagnostic testing performed on breath samples, specifically optimizing the pneumatics and fluid dynamics of a breath test system to be able to perform accurate sample collection and accurate sample measurement of a breath sample.

### BACKGROUND

Breath analysis devices that isolate and measure one section of a breath, usually have a disposable patient interface and an instrument to draw the sample from the patient interface and analyze the sample. It is necessary for the breath being drawn from the patient to travel through various componentry in both the patient interface and the instrument, such as tubing, connectors, valves, filters and sensors. It is desired however that the different constituent parts of the breath sample (for example, the start, middle and end of exhalation and inspiration) travel through the system as columns of different gas sections, each column after of the prior column, and with the boundary between neighboring columns taking the form of a discrete boundary line, rather than a boundary zone or area. The system should be designed so that gases from neighboring sections do not intermix, and there is a boundary line and not a boundary area. One way to accomplish this is to have a narrow cross section fluid pathway throughout the system. The cross section cannot be too resistive however because of other competing design constraints, such as constant sampling flow rates, turbulence, drag and other factors. A proper system balances the need for a narrow flow pathway channel with the need for minimal resistance to achieve the final desired results.

If the boundary between two gas sections traveling through the system can be a discrete line, than the section of interest of the breath, for example the end of exhalation, assuming it can be captured and isolated, can theoretically be measured in its entirety without worry that the front and back end could be contaminated with other breath sections. Another option is to measure only the very center of the section of interest, for example, discarding 25% of the beginning of the section and 25% of the end of the section, and only analyzing the middle 50% of the section. This would avoid using the part of the sample at the front and back ends that could be subject to contamination because of the boundary area, and this type of system would be theoretically capable of measuring pure end-tidal gas from the mid-section of the end-tidal sample. However, the system required to collect and measure breath samples operates in a substantially dynamic external and internal environment, and there are variable conditions difficult to recognize and control, and therefore it is best to avoid the mixing altogether if possible. If a sample is measured that includes boundaries that are mixed with other gases, the result will likely be contaminated and will either be diluted because it is mixed with a gas that has a higher content of ambient air then the gas section of interest, or conversely concentrated with the gas under investigation. Avoidance of the mixing assures a true, pure, accurate reading of the gas under investigation. The same disclosure and principles applies to other analytes in the breath, including non-gaseous analytes, and applies to measuring analytes from gas from different sections in the bronchial tree, for a host of clinical conditions and syndromes. End-tidal breath testing is used herein for exemplary purposes.

The solution to the problem of mixing is using novel, not before used, features in the components in the fluid path in order to maintain an appropriate cross section throughout all the componentry of the system, as described in the subsequent figures.

### BRIEF DESCRIPTION OF THE FIGURES

FIG. 1 schematically describes an overview including an instrument and a removably attachable patient interface.

FIG. 1a shows the system of FIG. 1 with the patient gas sample collection pathway active.

FIG. 1b shows the system of FIG. 1 with the ambient gas and gas analysis pathway active.

FIG. 2 schematically describes sections of respiration gas traveling through Detail A of the patient interface shown in FIG. 1.

FIG. 3 graphically describes the signal response from a breath analyte sensor with respect to time when measuring gas from a breath, and shows the improvement in measurement accuracy of embodiments over the prior art.

FIG. 4 shows a cross section side view of a filter used in a prior art patient interface for breath measurement.

FIG. 5 shows the filter of FIG. 4 with sections of exhaled gas flowing through it, showing a theoretical uniform flow profile through the filter, which in reality would not exist.

FIG. 6 shows the filter of FIG. 4 with sections of exhaled gas flowing through it, and mixing with other sections due to the volume expansion, as what would occur in reality with the prior art.

FIG. 7 is a cross sectional side view of a new filter including a concentric hydrophilic filter and a normal-to-flow hydrophobic filter.

FIG. 8 is a cross sectional side view of a new filter including axially straight concentric filters positioned in a straight sections of a curved flow channel filtration cartridge.

FIG. 9 is a hidden line front view of a nosepiece at the patient end of the patient interface, showing a constant size of the gas flow channel throughout this section.

FIG. 10 is a isometric view of a prior art nosepiece of a conventional nasal cannula device, showing the expanding gas flow channel throughout this section compared to the tubing connected to the nosepiece.

FIG. 11 is a schematic of the instrument showing zero dead-space pincher valves in the gas flow pathway.

FIG. 12 is a schematic of the instrument showing a valve-less gas flow pathway between the patient interface connection and the analyte sensor.

FIG. 13 shows a schematic drawing of a pneumatic system that splits the gas drawn from the patient into two pathways, one pathway for measuring the breathing signal and one pathway for measuring the amount of analyte in question in the breath, the latter pathway devoid of valves except the inlet valve.

FIG. 14 shows the system of FIG. 13 when the system is flushing the breathing signal sensor path.

FIG. 15 shows the system of FIG. 13 when the system is flushing the bypass path of the analyte measurement path.

FIG. 16 shows the system of FIG. 13 when the system is moving the analyte gas sample from the analyte pathway to the analyte sensor.

### DETAILED DESCRIPTION

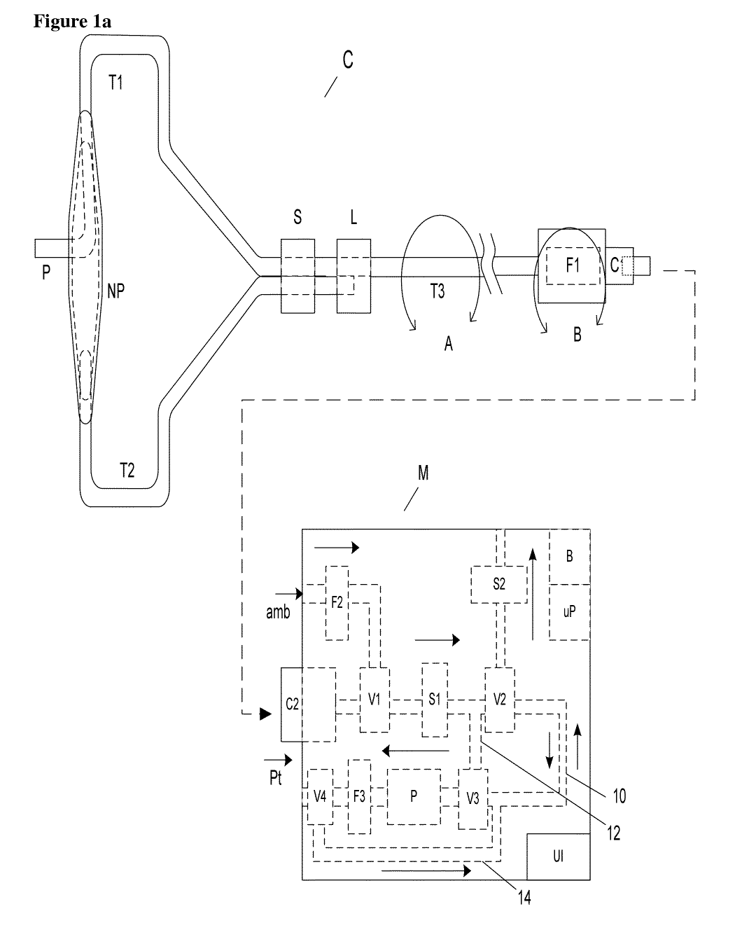

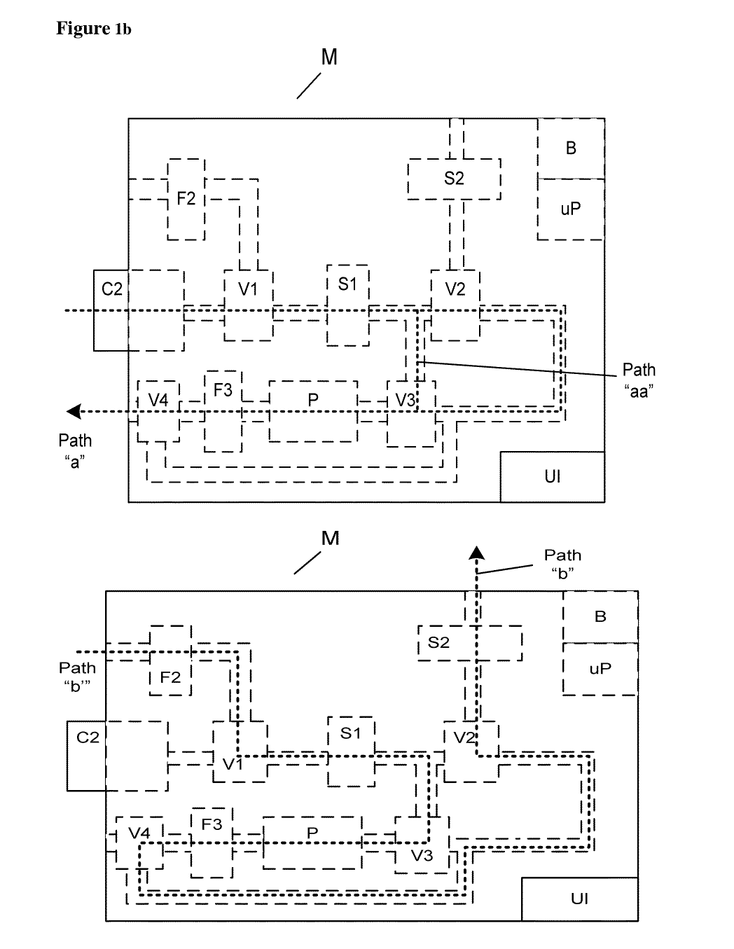

In FIG. 1 the overall system is described, which includes a patient interface C and instrument M. In the case described, the patient interface is a nasal cannula, however other types of patient interfaces and sampling cannula can be used, such as oral cannula, tracheal cannula, bronchial cannula, mouthpieces, mainstream collection adaptors, masks, and others. The cannula includes a nosepiece NP, a nasal prong P, a fluid flow path tube T1 on one side, and a non-flow path tube T2 on the other side to help hold the cannula to the face, and a connector C to connect to the instrument M. The connector includes a filter F1 or filters to filter humidity and bacteria from the patient which would otherwise harm the instrument and sensors. The instrument includes an inlet connector C2 for cannula attachment, an inlet value V1 to switch between gas from an ambient inlet amb and patient inlet Pt, a filter at the ambient inlet F2, a breathing pattern sensor S1 to query the breathing pattern of the gas from the patient, a sample tube 10 to contain the sample which is to be analyzed, an inlet and outlet valve V2 and V3 respectively to the sample tube, a bypass tube 12 to divert other gases around the gas sample in the sample tube, a push tube 14 to push the gas in the sample tube to the gas composition sensor S2, a pump P to draw the sample from the patient and optionally to push the sample to the gas composition sensor, a pump outlet filter F3 to protect the system from particulate stemming from the pump, a gas composition sensor S2, a valve V4 to control whether the pump is drawing from the patient or pushing the sample to the gas composition sensor. The instrument may include a battery B for operation, a microprocessor uP for control functions and other functions, and a user interface UI.

In FIG. 1a the instrument's gas flow path “a” is shown when a gas is being collected from the patient and either filling, path “a” or bypassing the sample tube 10, path “aa ”. In FIG. 1b the gas flow path “b” is shown when the sample is being diverted to the gas composition sensor.

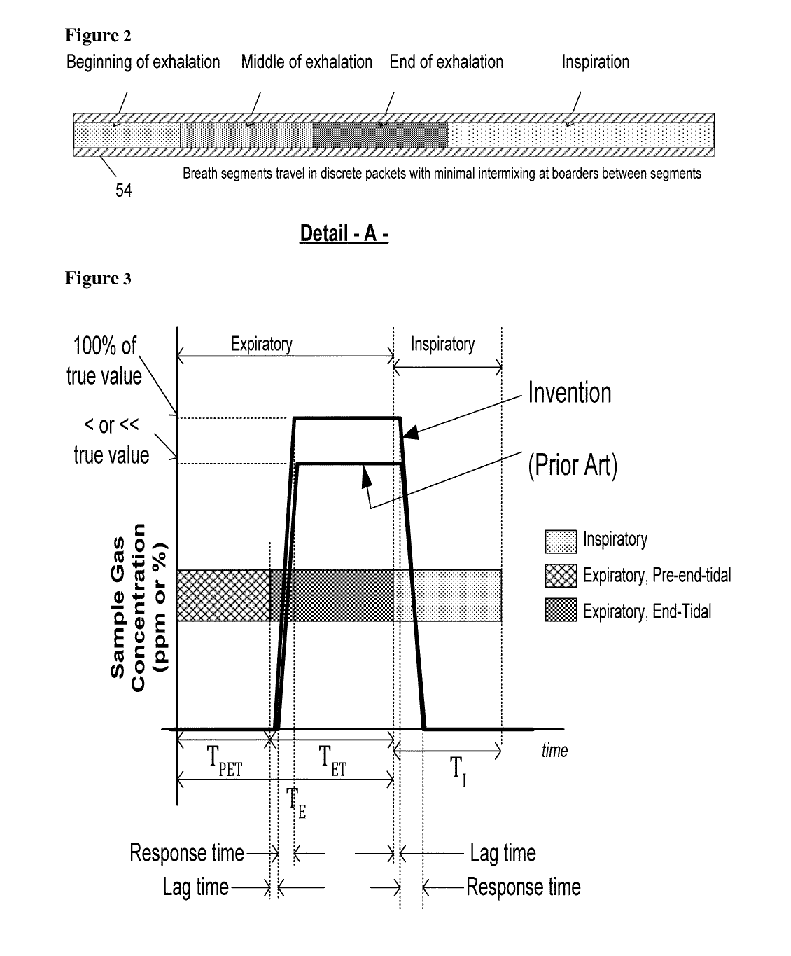

In FIG. 2 a section of the sampling pathway is shown from the tube section T3 from the patient interface shown in FIG. 1. Different sections of gas being drawn from the patient are shown traveling through the cannula 54. As can be seen, there are marked delineations between the different sections, rather than mixed transition zones. The breath gas segments are traveling in discrete packets with minimal or negligible intermixing at the borders. This is the gas flow behavior enabled by some embodiments and which is desired in a breath gas analysis system which used to measure gas in a certain section of the patient's breath. The sampling pathway diameter or effective diameter is typically 0.010″ to 0.080″, and preferably 0.020″ to 0.060″ and most preferably 0.030″ to 0.040″. These diameters or effective diameters are maintained throughout the system, and are chosen to balance competing requirements of minimal flow resistance and columnar flow anti-mixing behavior in the flow path.

In FIG. 3 the gas composition of a single breath is graphed, with amplitude on the vertical axis and one breath period on the horizontal axis. The graph shows two cases; a gas composition measurement using the prior art, and a gas composition measurement using some embodiments. In the prior art, the measured gas composition amplitude is lower compared to that of some embodiments described herein, because in the prior art example, the gas sample became diluted by traveling through the various dead-space volumes throughout the system. In the curve representing the present disclosure, the signal amplitude reaches its maximum potential because the gas sample does not get mixed, is non-contaminated and remains pure, and therefore the sensor signal can be correlated to the true gas composition for an accurate diagnostic assessment.

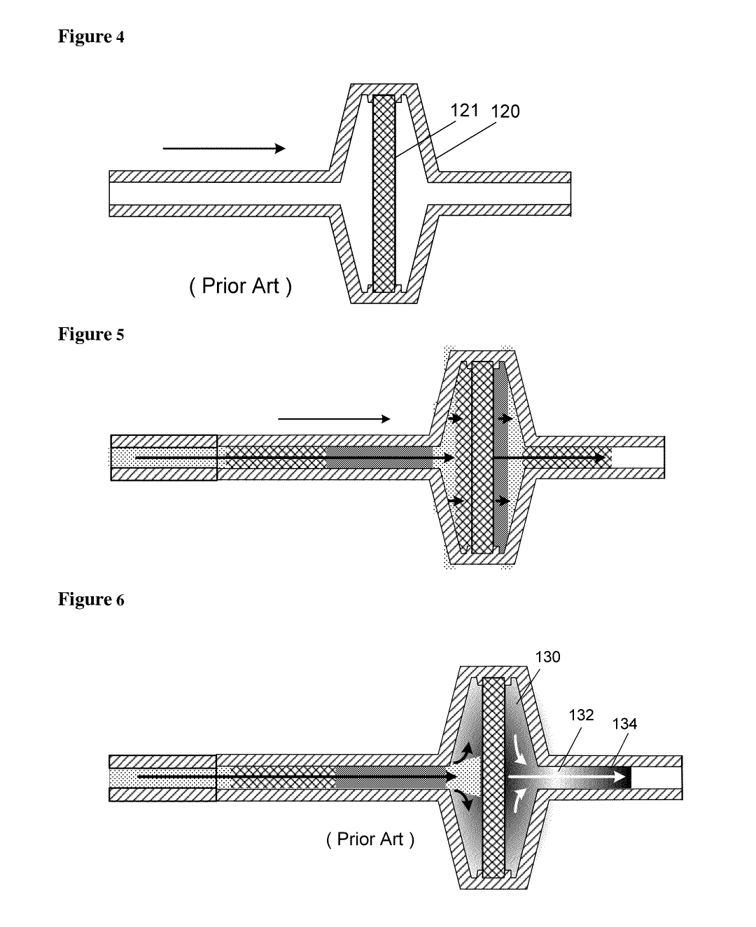

FIGS. 4-6 describe a cross section of an example of a component in the gas sampling pathway, for example a filter used in the prior art. In this example, the filter adds too much dead-space to the system and allows the gas to mix, resulting in the prior art gas composition curve shown in FIG. 3. A filter may be required in a gas analysis system for filtering humidity and bacteria. FIG. 4 shows the gas pathway tubing T3 on the inlet side of the filter 120, the filter element 121 which is a disk type filter, and the gas pathway conduit on the outlet side of the filter. As shown in FIG. 5, the gas sampling pathway on the inlet side of the filter contains different sections of the breath gas adjacent to one another end to end. The gas travels in discrete packets, for example, beginning of exhalation 112, end of exhalation 114 and inspiration 110. It might be thought that the gas enters the filter, expands into the larger cross sectional flow profile of the filter, but still travels through the filter with a linear flow profile and maintains the discrete borders between gas sections as shown in the filter section of FIG. 5. However, in reality, this does not occur. Rather, as shown in FIG. 6 the gas sections mix with one another in the filter as well as mix with the baseline gas that was in the filter before the patient gas enters the filter. The actual gas mixing behavior that really occurs is. The gas does not travel through the filter in a linear flow profile, but rather in a non-linear profile, which lends to intermixing of gases 130 from different sections of the breath inside the filter. The result is that on the outlet side of the filter, the borders between the different gas sections are now blurred, and there is a mixed gas zone between the different gas sections, and the pre-end-tidal gas is contaminated 132 and the end-tidal gas is contaminated 134. In addition, under certain system dynamics and dimensional conditions, the filter volume can be too large for a certain section of breath gas. For example, if the section of interest of gas is 0.X ml, and the filter volume is X.0, then the section of interest gas occupies only 10% of the filter volume, which leads to the possibility of mixing with other gases by diffusion and other gas mixing principles. Depending on the prevailing conditions which are dynamic, the entire gas section of interest can be diluted, concentrated or otherwise contaminated with other gases.

FIG. 7 shows a low dead-space filtration system to filter out the humidity and bacteria from the patient. In this example, the filter does not add dead-space to the system and thus prevents the gas from mixing, resulting in the improvement over the prior art shown on the gas composition curve in FIG. 3. A tubular hydrophilic filter 60 may be placed concentrically on the inner wall of the gas flow path inside a filter housing 50 of the cannula connector C1. The filter 60 may be tacked in place with adhesive 58, and joined with the cannula tubing 54 with the aid of a strain relief tube 56. A second stage hydrophilic filter 62 may also be used and be placed in the flow path and substantially normal to the flow path to prevent moisture from accumulating on the filter and to filter out bacteria. The combination of filters will filter out bacteria from passing through the filter since the water vapor will condensate out of suspension and form particulate water which will accumulate along the walls of the filter area. The bacteria will attach to the particulate water and thus will not travel through the second stage filter. Therefore, the second stage filter can be of a higher micron pore size than what is normally used to filter bacteria. For example a 1-5 micron filter will be sufficient, rather than the normally used 0.2 micron filter to filter out bacteria. A 0.2 micron filter, if used in the small gas flow channel which is needed to prevent mixing, would create substantially high flow resistance and substantially increases the pressure head rating of the pump employed by the system, or makes in more difficult to draw air from the patient. The second stage filter also serves to filter out larger molecules such as gases that may be harmful to the instrument and sensor such as aldehydes or ketones. This humidity filter arrangement may be capable of extracting from the flow path and storing 0.001 ml of water which provides the capacity to filter humidity from the patient for up to 5 hours of operation. When placed on the machine end of the sampling cannula, by the time breath gas travels from the patient to the filter, most water particles and molecules have contacted the wall of the cannula, and depending on the surface characteristics, migrates down the remaining length of the cannula along the wall, such that by the time the water reaches the tubular filter, it is already along the wall and easily is absorbed by the filter. In addition, the filter length can be such that, if the water particles or molecules are in the gas stream, due to time of flight, they will certainly contact the filter media before exiting the filter area.

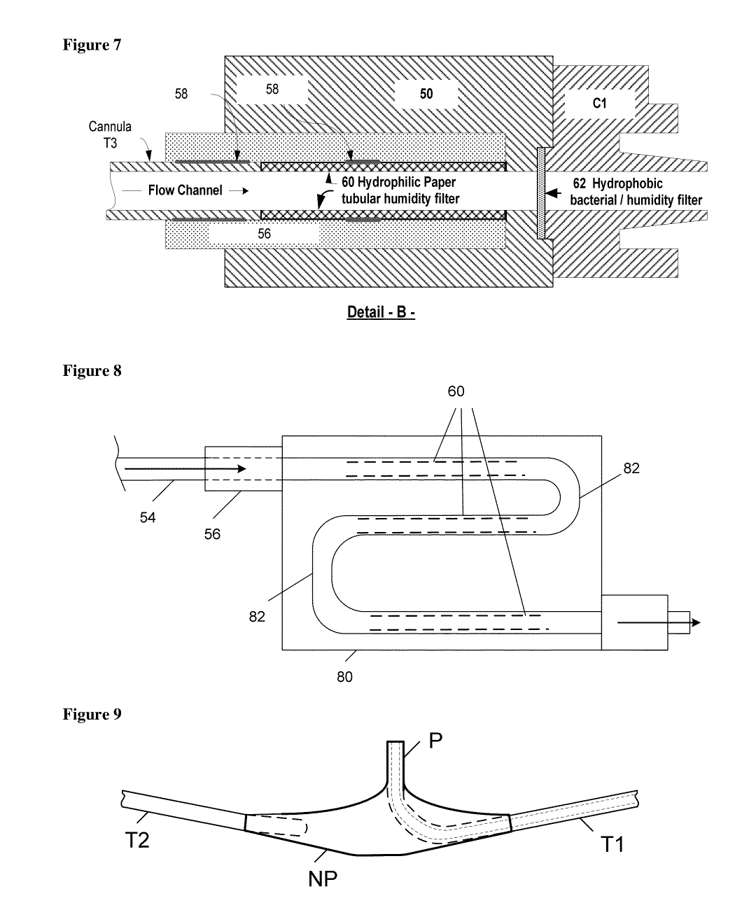

FIG. 8 describes an alternative in line humidity filter 80, in which the gas flow path is designed to make one or more bends or turns 82. Concentric hydrophilic filter elements 60 may be placed in straight sections of the filter 80. The bends will encourages water particles or molecules or vapor to impinge on the flow path wall in the bend area, which maximizes the chance that the water will contact the hydrophilic filter media. This filter arrangement adds no additional flow resistance to the system, and adds no unnecessary dead-space, yet provides effective humidity filtration.

FIG. 9 describes a nosepiece NP at the patient end of a nasal sampling cannula, with a flow path tube T1 attached to one end of the nosepiece and in communication with a nasal prong P, and a non-flow path tube T2 attached to the other end of the nosepiece to help secure the assembly to the patient's face. A compliant nosepiece section NP is included to help position the prong under the nose, and join the tubes that secure the cannula to the patient's face. The flow path tube and nasal prong may be a contiguous section of tubing of the correct inner diameter. The compliant nosepiece is bulbous to allow a generous curvature of the contiguous section of tubing, to provide cushioning and comfort, and to avoid kinks and obstructions. The gas flow path cross section remains constant and is devoid of enlarged sections and dead-space volumes, and therefore prevents any mixing behavior.

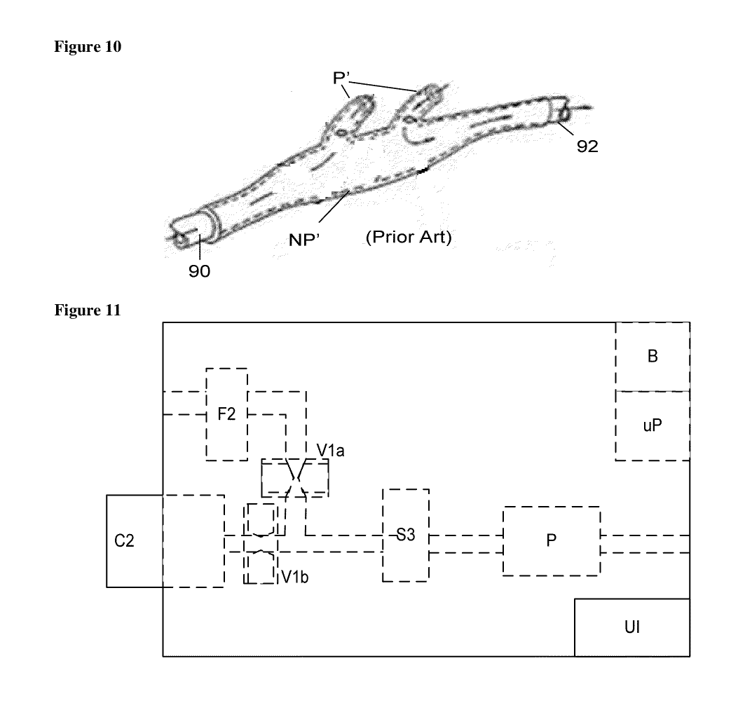

In contrast to the nosepiece shown in FIG. 9, FIG. 10 describes a nosepiece common in the prior art. This prior art nosepiece, and associated tubing and nasal prong assembly possesses a dead-space volume in the flow path. As described in the filter example, this volume will allow mixing and contamination of the section of the breath gas that is being targeted for measurement. FIG. 9 in contrast is a design which completely avoids this dead-space.

FIG. 11 describes an alternative instrument in which the control valve to switch the inlet gas from patient gas to ambient gas is a pair of zero-dead-space pinch style valves rather than a 3-way solenoid valve which inherently has some amount of dead-space. Gas from the patient enters from connector C2, gas for ambient enters via the ambient inlet filter F2, incoming gas travels through sensor S3, drawn by the pump P. The pincher valves, V1a and V1b work in unison to pinch and close one of the available inlet pathways. Valve Via is shown closed, closing the ambient inlet pathway, and Valve V1b is open, allowing the system to draw in air from the patient. Tubing of the desired narrow cross section ID passes through valve V1b, so that there is no opportunity for dead-space in the gas pathway to cause the gas to mix and become contaminated. The pincher valve is not additive to the volume of the system, whereas most solenoid valve designs in which the gas travels through the inner workings of the valve mechanism add some amount of dead-space to the system, which in this clinical application may be detrimental to accuracy for mixing related reasons.

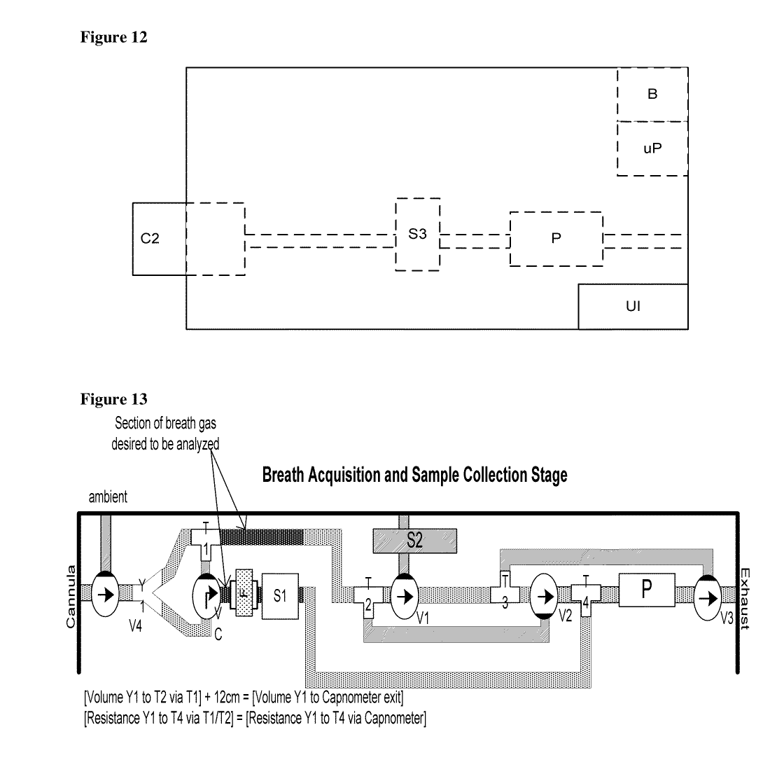

In addition, FIG. 11 describes an alternative configuration in which Sensor S3 serves two functions; (1) breathing pattern measurement used to find and target an acceptable breath for measurement, and (2) gas composition analysis of the gas in question. In this case the sensor is a fast sensor capable of responding to the gas relatively quickly, for example within 0.2 seconds. This configuration avoids the need to separate out the desired gas section from the other sections for subsequent transfer to the separate gas composition sensor. FIG. 12 describes an alternate configuration in which the system does not include an ambient gas sampling pathway and therefore does not require a control valve to switch between patient gas and ambient gas, thereby avoiding the potential for dead-space-related gas mixing in a valve.

FIGS. 13-16 describe an alternative configuration for a minimal dead-space design to prevent intermixing of gases, referred to as a split flow design. The incoming flow from the patient is spilt into two paths. The lower path goes through valve VC and the breath pattern sensor S1, tee T4, through the pump and through valve V3 to the exhaust The upper path goes around the breath pattern sensor S1 to either valve V1 to the sample collection tube, or around V1 and the sample collection tube via tee T2 to valve V2 and also through the pump and out the exhaust through valve V3. This configuration is useful when the breath pattern sensor is of a type that has a substantial enough dead-space that has the potential to mix gases. The resistances, speed and travel distances of the upper and lower pathways are carefully balanced, understood and controlled, such that the time that the beginning and end of the desired gas sample reaches V1 can be predicted with accuracy based on the timing of the sample travelling through sensor S1. It should be noted that valve V4 and Valve V1 which are in the flow pathway of the sample that will eventually get measured, can be pincher valves rather than solenoid valves, to prevent mixing due to valve dead-space. FIG. 13 shows the system during breath sample acquisition, indicating schematically a section of breath gas that is desired to be captured and analyzed. This section of gas bifurcates into two sections at the Y connector Y1, one section traveling in the lower path, and one section traveling in the upper path. Both sections inherently have the same concentration of analyte that is intended to be measured. In the lower pathway, the sample may get diluted by valves and the sensor S2, however that is of no concern. The lower pathway is being used only to understand the timing of the sample in the upper pathway.

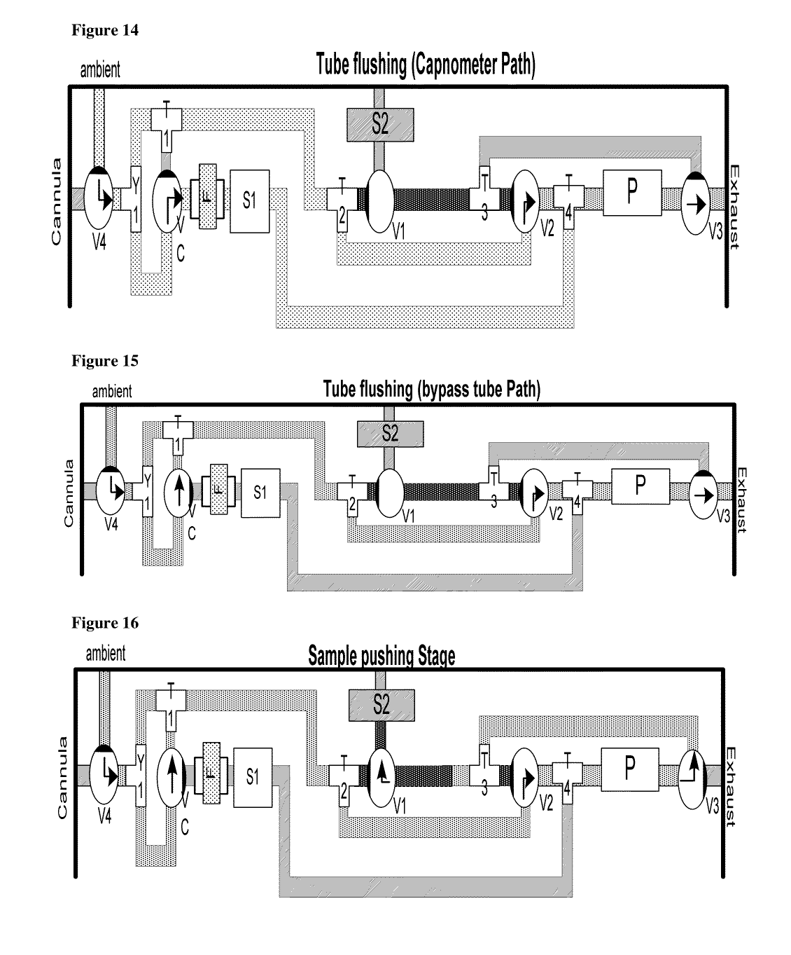

In FIG. 14 the desired sample of gas has been shunted into the sample tube between valve V1 and Valve V2, and isolated there by switching the porting of those valves once the sample is in place. Next, the ambient inlet can be opened for the purpose of flushing residual patient gases out of the lower pathway of the system so there is no chance of contamination of the sample. In FIG. 15 the upper pathway and bypass tube are flushed by ambient air also to prevent any chance of sample contamination. Next, shown in FIG. 16, the sample can be diverted out of its holding place between valve V1 and V2 and to the sensor S2 for analysis.

It should be noted that in the embodiments described, the pneumatic system may comprise a separate breathing pattern sensor, and a separate breath analyte composition sensor, however it is contemplated that in the embodiments, the two functions can be handled by the same sensor. The section of gas desired to be measured can be an end-tidal section of gas, a deep alveolar sample of gas, a lower airway sample of gas, a middle airway sample of gas, or an upper airway sample of gas. The system described in this invention may be used for measuring, monitoring, estimating or assessing various analytes in the breath, and can be used to assess or diagnose various diseases, disorders, syndromes.

What is claimed is:

1. An apparatus for measuring a breath analyte comprising:
a nasal prong;
a nosepiece comprising an inlet and an outlet;
a first flow channel extending from the nasal prong to the nosepiece inlet;
a second flow channel within the nosepiece and extending from the nosepiece inlet to the nosepiece outlet, wherein the cross-section of the second flow channel is substantially constant between the nosepiece inlet and nosepiece outlet; and
a third flow channel extending from the nosepiece outlet to a breath measurement system.

2. The apparatus of claim 1, wherein all three flow channels form part of a continuous tube.

3. The apparatus of claim 2, wherein the continuous tube comprises a cross-sectional diameter between 0.01″ and 0.06″.

4. The apparatus of claim 3, wherein the cross-sectional diameter is between 0.02″ and 0.04″.

5. The apparatus of claim 1, further comprising a support connected to the nosepiece on an opposite side of the nosepiece outlet, wherein the support is not fluidly connected to the second flow channel.

6. The apparatus of claim 5, wherein the support and the third channel are connected, and wherein the support, the third channel, and the nosepiece comprise a loop.

7. The apparatus of claim 6, further comprising a connection to couple the support and the third channel, and wherein the loop comprises the connection.

8. The apparatus of claim 1, wherein the first, second, and third flow channels are configured for a liner gas flow profile there through.

9. The apparatus of claim 8, wherein the flow channels' cross-section diameters are between 0.01″ and 0.06″.

10. The apparatus of claim 9, wherein the flow channels' cross-section diameters are between 0.02″ and 0.04″.

11. A method for measuring a breath analyte comprising:
inserting a nasal prong into a patient, wherein
a first flow channel extends from the nasal prong to the nosepiece inlet, wherein
a second flow channel extends from the nosepiece inlet to a nosepiece outlet, wherein the cross-section of the second flow channel is substantially constant between the nosepiece inlet and nosepiece outlet, and wherein
a third flow channel extends from the nosepiece outlet to a breath measurement system.

12. The method of claim 11, wherein all three flow channels form part of a continuous tube.

13. The method of claim 12, wherein the continuous tube comprises a cross-sectional diameter between 0.01″ and 0.06″.

14. The method of claim 13, wherein the cross-sectional diameter is between 0.02″ and 0.04″.

15. The method of claim 11, further comprising a support connected to the nosepiece on an opposite side of the nosepiece outlet, wherein the support is not fluidly connected to the second flow channel.

16. The method of claim 15, wherein the support and the third channel are connected, and wherein the support, the third channel, and the nosepiece comprise a loop.

17. The method of claim 16, further comprising a connection to couple the support and the third channel, and wherein the loop comprises the connection.

18. The method of claim 11, wherein the first, second, and third flow channels are configured for a liner gas flow profile there through.

19. The method of claim 18, wherein the flow channels' cross-section diameters are between 0.01″ and 0.06″.

20. The method of claim 19, wherein the flow channels' cross-section diameters are between 0.02″ and 0.04″.
